(19) United States
(12) Patent Application Publication
(10) Pub. No.: US 2010/0028385 A1
(43) Pub. Date: Feb. 4, 2010

(54) TREATMENT OF EXCESS CERUMEN SECRETION

(75) Inventor: Claude L. NASSIF, Los Angeles, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 12/185,749

(22) Filed: Aug. 4, 2008

(51) Int. Cl. A61K 39/08; A61P 43/00

(52) U.S. Cl. 424/239.1

(57) ABSTRACT

Presently descibed are methods useful in treating outer ear conditions generally associated with the secretion of cerumen. The methods comprise the step of locally administering a therapeutically effective amount of neurotoxin such as a botulinum toxin to the ear canal of a patient. The conditions to be treated are generally associated with excess secretion of cerumen or the build-up of cerumen in the ear canal of a patient. Such conditions can include, but are not limited to, conductive hearing loss, pain and cough, for example.

### FIELD OF THE INVENTION

The present invention describes methods of treating conditions resulting from excessive secretion and/or build-up of cerumen using a neurotoxin.

### BACKGROUND

Cerumen is produced by the cerumen glands, which are only found in the skin of the ear canals. The cerumen in the ear canals acts to protect the eardrums and acts as a trap for foreign debris that may enter and damage ears. The cerumen sequesters foreign debris and keeps it from reaching the eardrum.

Old cerumen in an ear canal should naturally work its way out or the ear canal as new cerumen is secreted. Occasionally, the cerumen glands can produce excess cerumen which cannot naturally escape the ear canal. This results in partial, or even in some circumstances full, blockage of the ear canal. Other times, cerumen builds up as a result of inappropriate ear canal intervention. Although a light cleaning of the ear canal can be accomplished using a cotton swab, this method can push cerumen farther into the ear canal and lead to canal blockage.

Other items that can alter natural cerumen migration are hearing aids. Hearing aids are placed into the ear canal of a person in need thereof and help that person hear sounds they ordinary would not or might not. Although helpful, a hearing aid, when inserted into the ear canal, can push cerumen farther into the ear, similar to compaction resulting from improper use of a cotton swab. Additionally, a hearing aid can block the migration of cerumen altogether, thereby causing build up of cerumen between a hearing aid and the eardrum. If this cerumen is not properly excavated, the hearing aid user could be susceptible to complications.

Why people experience excess cerumen secretion is not well understood. However, ear surgery can occasionally result in excess cerumen secretion. People can, in fact, develop excess secretion in one ear or the other, or even differing amounts of secretion in each ear. Regardless, a method needs to be developed wherein this excess secretion can be treated and/or eliminated, hence reducing and/or eliminating any build-up resulting therefrom. Methods will be discussed herein which involve the administration of a toxin to the ear canal of a patient, thereby treating excess production and/or build-up of cerumen.

The genus Clostridium has more than one hundred and twenty seven species, grouped according to their morphology and functions. The anaerobic, gram positive bacterium Clostridium botulinum produces a potent polypeptide neurotoxin, botulinum toxin, which causes a neuroparalytic illness in humans and animals referred to as botulism. The spores of Clostridium botulinum are found in soil and can grow in improperly sterilized and sealed food containers of home based canneries, which are the cause of many of the cases of botulism. The effects of botulism typically appear 18 to 36 hours after eating the foodstuffs infected with a Clostridium botulinum culture or spores. The botulinum toxin can apparently pass unattenuated through the lining of the gut and shows a high affinity for cholinergic motor neurons. Symptoms of botulinum toxin intoxication can progress from difficulty walking, swallowing, and speaking to paralysis of the respiratory muscles and death.

About 50 picograms of a commercially available botulinum toxin type A (purified neurotoxin complex) has an LD₅₀ in mice (i.e. 1 unit). One unit of BOTOX® (botulinum toxin type A, Allergan, Inc., Irvine, Calif.) contains about 50 picograms (about 56 attomoles) of botulinum toxin type A complex. Interestingly, on a molar basis, botulinum toxin type A is about 1.8 billion times more lethal than diphtheria, about 600 million times more lethal than sodium cyanide, about 30 million times more lethal than cobra toxin and about 12 million times more lethal than cholera (Singh, Critical Aspects of Bacterial Protein Toxins, pages 63-84 (chapter 4) of Natural Toxins II, edited by B R. Singh et al., Plenum Press, New York (1976) (where the stated LD₅₀ of botulinum toxin type A of 0.3 ng equals 1 U is corrected for the fact that about 0.05 ng of BOTOX® equals 1 unit)). One unit (U) of botulinum toxin is defined as the LD₅₀ upon intraperitoneal injection into female Swiss Webster mice weighing 18 to 20 grams each. (Available from Allergan, Inc., of Irvine, Calif. under the tradename BOTOX® in 100 unit vials).

Seven generally immunologically distinct botulinum neurotoxins have been characterized, these being respectively botulinum neurotoxin serotypes A, B, C₁, D, E, F and G, each of which is distinguished by neutralization with type-specific antibodies. The different serotypes of botulinum toxin vary in the animal species that they affect and in the severity and duration of the paralysis they evoke. For example, it has been determined that botulinum toxin type A is 500 times more potent, as measured by the rate of paralysis produced in the rat, than is botulinum toxin type B. Additionally, botulinum toxin type B has been determined to be non-toxic in primates at a dose of 480 U/kg which is about 12 times the primate LD₅₀ for botulinum toxin type A (Moyer E et al., Botulinum Toxin Type B: Experimental and Clinical Experience, being chapter 6, pages 71-85 of “Therapy With Botulinum Toxin”, edited by Jankovic, J. et al. (1994), Marcel Dekker, Inc.).

Regardless of serotype, the molecular mechanism of toxin intoxication appears to be similar and involve at least three steps or stages. In the first step of the process, the toxin binds to the presynaptic membrane of the target neuron through a specific interaction between the heavy chain (the H chain or HC), and a cell surface receptor. The receptor is thought to be different for each type of botulinum toxin and for tetanus toxin. The carboxyl end segment of the HC appears to be important for targeting of the botulinum toxin to the cell surface.

In the second step, the botulinum toxin crosses the plasma membrane of the target cell. The botulinum toxin is first engulfed by the cell through receptor-mediated endocytosis, and an endosome containing the botulinum toxin is formed. The toxin then escapes the endosome into the cytoplasm of the cell. This step is thought to be mediated by the amino end segment of the HC, the HN, which triggers a conformational change of the toxin in response to a pH of about 5.5 or lower. Endosomes are known to possess a proton pump which decreases intra-endosomal pH. The conformational shift exposes hydrophobic residues in the toxin, which permits the botulinum toxin to embed itself in the endosomal membrane. The botulinum toxin (or at least the light chain of the botulinum) then translocates through the endosomal membrane into the cytoplasm.

The last step of the mechanism of botulinum toxin activity appears to involve reduction of the disulfide bond joining the heavy chain, H chain, and the light chain, L chain. The entire toxic activity of botulinum and tetanus toxins is contained in the L chain of the holotoxin; the L chain is a zinc (Zn²⁺) endopeptidase which selectively cleaves proteins essential for recognition and docking of neurotransmitter-containing vesicles with the cytoplasmic surface of the plasma membrane, and fusion of the vesicles with the plasma membrane. Tetanus neurotoxin, botulinum toxin types B, D, F, and G cause degradation of synaptobrevin (also called vesicle-associated membrane protein (VAMP)), a synaptosomal membrane protein. Most of the VAMP present at the cytoplasmic surface of the synaptic vesicle is removed as a result of any one of these cleavage events. Botulinum toxin serotype A and E cleave SNAP-25. Botulinum toxin serotype C₁ was originally thought to cleave syntaxin, but was found to cleave syntaxin and SNAP-25. Each of the botulinum toxins specifically cleaves a different bond, except botulinum toxin type B (and tetanus toxin) which cleave the same bond. Each of these cleavages block the process of vesicle-membrane docking, thereby preventing exocytosis of vesicle content.

Although all the botulinum toxins serotypes apparently inhibit release of the neurotransmitter acetylcholine at the neuromuscular junction, they do so by affecting different neurosecretory proteins and/or cleaving these proteins at different sites. For example, botulinum types A and E both cleave the 25 kiloDalton (kD) synaptosomal associated protein (SNAP-25), but they target different amino acid sequences within this protein. Botulinum toxin types B, D, F and G act on vesicle-associated protein (VAMP, also called synaptobrevin), with each serotype cleaving the protein at a different site. Finally, botulinum toxin type C₁ has been shown to cleave both syntaxin and SNAP-25. These differences in mechanism of action may affect the relative potency and/or duration of action of the various botulinum toxin serotypes. Apparently, a substrate for a botulinum toxin can be found in a variety of different cell types. See e.g. Biochem J 1;339 (pt 1): 159-65:1999, and Mov Disord, 10(3):376:1995 (pancreatic islet B cells contains at least SNAP-25 and synaptobrevin).

The molecular weight of the botulinum toxin protein molecule, for all seven of the known botulinum toxin serotypes, is about 150 kD. Interestingly, the botulinum toxins are released by Clostridial bacterium as complexes comprising the 150 kD botulinum toxin protein molecule along with associated non-toxin proteins. Thus, the botulinum toxin type A complex can be produced by Clostridial bacterium as 900 kD, 500 kD and 300 kD forms. Botulinum toxin types B and C, are apparently produced as only a 700 kD or 500 kD complex. Botulinum toxin type D is produced as both 300 kD and 500 kD complexes. Finally, botulinum toxin types E and F are produced as only approximately 300 kD complexes. The complexes (i.e. molecular weight greater than about 150 kD) are believed to contain a non-toxin hemaglutinin proteins and a non-toxin and non-toxic nonhemaglutinin protein. These two non-toxin proteins (which along with the botulinum toxin molecule comprise the relevant neurotoxin complex) may act to provide stability against denaturation to the botulinum toxin molecule and protection against digestive acids when a botulinum toxin is ingested. Additionally, it is possible that the larger (greater than about 150 kD molecular weight) botulinum toxin complexes may result in a slower rate of diffusion of the botulinum toxin away from a site of injection of a botulinum toxin complex.

Botulinum toxin type A can be obtained by establishing and growing cultures of Clostridium botulinum in a fermenter and then harvesting and purifying the fermented mixture in accordance with known procedures. All the botulinum toxin serotypes are initially synthesized as inactive single chain proteins which must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C₁, D and E are synthesized by nonproteolytic strains and are therefore typically unactivated when recovered from culture. Serotypes B and F are produced by both proteolytic and nonproteolytic strains and therefore can be recovered in either the active or inactive form. However, even the proteolytic strains that produce, for example, the botulinum toxin type B serotype only cleave a portion of the toxin produced. The exact proportion of nicked to unnicked molecules depends on the length of incubation and the temperature of the culture. Therefore, a certain percentage of any preparation of, for example, the botulinum toxin type B toxin is likely to be inactive, possibly accounting for the known significantly lower potency of botulinum toxin type B as compared to botulinum toxin type A. The presence of inactive botulinum toxin molecules in a clinical preparation will contribute to the overall protein load of the preparation, which has been linked to increased antigenicity, without contributing to its clinical efficacy.

High quality crystalline botulinum toxin type A can be produced from the Hall A strain of Clostridium botulinum with characteristics of ≦3×10⁷ U/mg, an A₂₆₀/A₂₇₈ of less than 0.60 and a distinct pattern of banding on gel electrophoresis. The known Shantz process can be used to obtain crystalline botulinum toxin type A, as set forth in Shantz, E. J., et al. (Properties and use of Botulinum toxin and Other Microbial Neurotoxins in Medicine, Microbiol Rev. 56: 80-99, 1992). Generally, the botulinum toxin type A complex can be isolated and purified from an anaerobic fermentation by cultivating Clostridium botulinum type A in a suitable medium. The known process can also be used, upon separation out of the non-toxin proteins, to obtain pure botulinum toxins, such as for example: purified botulinum toxin type A with an approximately 150 kD molecular weight with a specific potency of 1-2×10⁸ LD₅₀ U/mg or greater; purified botulinum toxin type B with an approximately 156 kD molecular weight with a specific potency of 1-2×10⁸ LD₅₀ U/mg or greater, and; purified botulinum toxin type F with an approximately 155 kD molecular weight with a specific potency of 1-2×10⁷ LD₅₀ U/mg or greater.

As with enzymes generally, the biological activities of the botulinum toxins (which are intracellular peptidases) are dependant, at least in part, upon their three-dimensional conformation. Thus, botulinum toxin type A is detoxified by heat, various chemicals surface stretching and surface drying. Additionally, it is known that dilution of a botulinum toxin complex obtained by the known culturing, fermentation and purification to the much, much lower toxin concentrations used for pharmaceutical composition formulation results in rapid detoxification of the toxin unless a suitable stabilizing agent is present. Dilution of the toxin from milligram quantities to a solution containing nanograms per milliliter presents significant difficulties because of the rapid loss of specific toxicity upon such great dilution. Since the botulinum toxin may be used months or years after the toxin containing pharmaceutical composition is formulated, the toxin can be stabilized with a stabilizing agent such as albumin and gelatin.

A commercially available botulinum toxin containing pharmaceutical composition is sold under the trademark BOTOX® (available from Allergan, Inc., of Irvine, Calif.). BOTOX® consists of a purified botulinum toxin type A complex, albumin and sodium chloride packaged in sterile, vacuum-dried form. The botulinum toxin type A is made from a culture of the Hall strain of Clostridium botulinum grown in a medium containing N-Z amine and yeast extract. The botulinum toxin type A complex is purified from the culture solution by a series of acid precipitations to a crystalline complex consisting of the active high molecular weight toxin protein and an associated hemagglutinin protein. The crystalline complex is re-dissolved in a solution containing saline and albumin and sterile filtered (0.2 microns) prior to vacuum-drying. The vacuum-dried product is stored in a freezer at or below −5° C. BOTOX® can be reconstituted with sterile, non-preserved saline prior to intramuscular injection. Each vial of BOTOX® contains about 100 units (U) of Clostridium botulinum toxin type A purified neurotoxin complex, 0.5 milligrams of human serum albumin and 0.9 milligrams of sodium chloride in a sterile, vacuum-dried form without a preservative.

To reconstitute vacuum-dried BOTOX®, sterile normal saline without a preservative, (0.9% Sodium Chloride Injection) is used by drawing up the proper amount of diluent in the appropriate size syringe. Since BOTOX® may be denatured by bubbling or similar violent agitation, the diluent is gently injected into the vial. For sterility reasons BOTOX® is preferably administered within four hours after the vial is removed from the freezer and reconstituted. During these four hours, reconstituted BOTOX® can be stored in a refrigerator at about 2° C. to about 8° C. Reconstituted, refrigerated BOTOX® has been reported to retain its potency for at least about two weeks.

It has been reported that botulinum toxin type A has been used in clinical settings as follows:

(1) about 75-125 units of BOTOX® per intramuscular injection (multiple muscles) to treat cervical dystonia;

(2) 5-10 units of BOTOX® per intramuscular injection to treat glabellar lines (brow furrows) (5 units injected intramuscularly into the procerus muscle and 10 units injected intramuscularly into each corrugator supercilii muscle);

(3) about 30-80 units of BOTOX® to treat constipation by intrasphincter injection of the puborectalis muscle;

(4) about 1-5 units per muscle of intramuscularly injected BOTOX® to treat blepharospasm by injecting the lateral pre-tarsal orbicularis oculi muscle of the upper lid and the lateral pre-tarsal orbicularis oculi of the lower lid;

(5) to treat strabismus, extraocular muscles have been injected intramuscularly with between about 1-5 units of BOTOX®, the amount injected varying based upon both the size of the muscle to be injected and the extent of muscle paralysis desired (i.e. amount of diopter correction desired);

(6) to treat upper limb spasticity following stroke by intramuscular injections of BOTOX® into five different upper limb flexor muscles, as follows:

(a) flexor digitorum profundus: 7.5 U to 30 U

(b) flexor digitorum sublimus: 7.5 U to 30 U

(c) flexor carpi ulnaris: 10 U to 40 U

(d) flexor carpi radialis: 15 U to 60 U

(e) biceps brachii: 50 U to 200 U. Each of the five indicated muscles has been injected at the same treatment session, so that the patient receives from 90 U to 360 U of upper limb flexor muscle BOTOX® by intramuscular injection at each treatment session; and

(7) to treat migraine, pericranial injected (injected symmetrically into glabellar, frontalis and temporalis muscles) injection of 25 U of BOTOX® has showed significant benefit as a prophylactic treatment of migraine compared to vehicle as measured by decreased measures of migraine frequency, maximal severity, associated vomiting and acute medication use over the three month period following the 25 U injection.

It is known that botulinum toxin type A can have an efficacy for up to 12 months (European J. Neurology 6 (Supp 4): S111-S1150:1999), and in some circumstances for as long as 27 months, when used to treat glands, such as in the treatment of hyperhydrosis. See e.g. Bushara K., Botulitum toxin and rhinorrhea, Otolaryngol Head Neck Surg 1996;114(3):507, and The Laryngoscope 109:1344-1346:1999. However, the usual duration of an intramuscular injection of BOTOX® is typically about 3 to 4 months.

The success of botulinum toxin type A to treat a variety of clinical conditions has led to interest in other botulinum toxin serotypes. Two commercially available botulinum type A preparations for use in humans are BOTOX® available from Allergan, Inc., of Irvine, Calif., and DYSPORT® available from Beaufour Ipsen, Porton Down, England. A Botulinum toxin type B preparation (MYOBLOC®) is available from Elan Pharmaceuticals of San Francisco, Calif.

A botulinum toxin has also been proposed for or has been used to treat skin wounds (U.S. Pat. No. 6,447,787), various autonomic nerve dysfunctions (U.S. Pat. No. 5,766,605), tension headache, (U.S. Pat. No. 6,458,365), migraine headache pain (U.S. Pat. No. 5,714,468), post-operative pain and visceral pain (U.S. Pat. No. 6,464,986), hair growth and hair retention (U.S. Pat. No. 6,299,893), psoriasis and dermatitis (U.S. Pat. No. 5,670,484), injured muscles (U.S. Pat. No. 6,423,319) various cancers (U.S. Pat. No. 6,139,845), smooth muscle disorders (U.S. Pat. No. 5,437,291), nerve entrapment syndromes (U.S. Published Patent Application 20030224019, filed Feb. 27, 2003), acne (WO 03/011333) and neurogenic inflammation (U.S. Pat. No. 6,063,768). Controlled release toxin implants are known (see e.g. U.S. Pat. Nos. 6,306,423 and 6,312,708) as is transdermal botulinum toxin administration (U.S. Published Patent Application No. 20040009180, filed Jul. 11, 2002), all herein incorporated by reference in their entirety.

It is known that a botulinum toxin can be used to weaken the chewing or biting muscle of the mouth so that self inflicted wounds and resulting ulcers can heal (Payne M., et al, Botulinum toxin as a novel treatment for self mutilation in Lesch-Nyhan syndrome, Ann Neurol September 2002;52(3 Supp 1):S157); permit healing of benign cystic lesions or tumors (Blugerman G., et al., Multiple eccrine hidrocystomas: A new therapeutic option with botulinum toxin, Dermatol Surg May 2003;29(5):557-9); treat anal fissure (Jost W., Ten years' experience with botulinum toxin in anal fissure, Int J Colorectal Dis September 2002;17(5):298-302, and; treat certain types of atopic dermatitis (Heckmann M., et al., Botulinum toxin type A injection in the treatment of lichen simplex: An open pilot study, J Am Acad Dermatol April 2002;46(4):617-9).

Additionally, a botulinum toxin may have an effect to reduce induced inflammatory pain in a rat formalin model. (Aoki K., et al, Mechanisms of the antinociceptive effect of subcutaneous Botox; Inhibition of peripheral and central nociceptive processing, Cephalalgia September 2003;23(7):649). Furthermore, it has been reported that botulinum toxin nerve blockage can cause a reduction of epidermal thickness. (Li Y, et al., Sensory and motor denervation influences epidermal thickness in rat foot glabrous skin, Exp Neurol 1997;147:452-462). Finally, it is known to administer a botulinum toxin to the foot to treat excessive foot sweating (Katsambas A., et al., Cutaneous diseases of the foot: Unapproved treatments, Clin Dermatol November-December 2002;20(6):689-699; Sevim, S., et al., Botulinum toxin-A therapy for palmar and plantar hyperhidrosis, Acta Neurol Belg December 2002;102(4):167-70), spastic toes (Suputtitada, A., Local botulinum toxin type A injections in the treatment of spastic toes, Am J Phys Med Rehabil October 2002;81(10):770-5), idiopathic toe walking (Tacks, L., et al., Idiopathic toe walking: Treatment with botulinum toxin A injection, Dev Med Child Neurol 2002;44(Suppl 91):6), and foot dystonia (Rogers J., et al., Injections of botulinum toxin A in foot dystonia, Neurology April 1993;43(4 Suppl 2)).

Tetanus toxin, as wells as derivatives (i.e. with a non-native targeting moiety), fragments, hybrids and chimeras thereof can also have therapeutic utility. The tetanus toxin bears many similarities to the botulinum toxins. Thus, both the tetanus toxin and the botulinum toxins are polypeptides made by closely related species of Clostridium (Clostridium tetani and Clostridium botulinum, respectively). Additionally, both the tetanus toxin and the botulinum toxins are dichain proteins composed of a light chain (molecular weight about 50 kD) covalently bound by a single disulfide bond to a heavy chain (molecular weight about 100 kD). Hence, the molecular weight of tetanus toxin and of each of the seven botulinum toxins (non-complexed) is about 150 kD. Furthermore, for both the tetanus toxin and the botulinum toxins, the light chain bears the domain which exhibits intracellular biological (protease) activity, while the heavy chain comprises the receptor binding (immunogenic) and cell membrane translocational domains.

Further, both the tetanus toxin and the botulinum toxins exhibit a high, specific affinity for gangliocide receptors on the surface of presynaptic cholinergic neurons. Receptor mediated endocytosis of tetanus toxin by peripheral cholinergic neurons results in retrograde axonal transport, blocking of the release of inhibitory neurotransmitters from central synapses and a spastic paralysis. Contrarily, receptor mediated endocytosis of botulinum toxin by peripheral cholinergic neurons results in little if any retrograde transport, inhibition of acetylcholine exocytosis from the intoxicated peripheral motor neurons and a flaccid paralysis.

Finally, the tetanus toxin and the botulinum toxins resemble each other in both biosynthesis and molecular architecture. Thus, there is an overall 34% identity between the protein sequences of tetanus toxin and botulinum toxin type A, and a sequence identity as high as 62% for some functional domains. (Binz T. et al., The Complete Sequence of Botulinum Neurotoxin Type A and Comparison with Other Clostridial Neurotoxins, J Biological Chemistry 265(16); 9153-9168:1990).

A common method of removing impacted cerumen involves washing out the ear canal with water, utilizing a syringe with a catheter attached, for example. This is referred to as ear canal irrigation. A small amount of alcohol, hydrogen peroxide, or other antiseptic can be added to the water that is to be flushed into and out of the ear canal. To minimize discomfort, water is kept close to body temperature. After the ear has been irrigated, antibiotic ear drops can be administered to the ear canal to protect the ear from infection. Exemplary antibiotics include, but are not limited to neomycin, polymyxin B, and hydrocortisone, for example.

Irrigation is typically contraindicated if the patient's eardrum is ruptured, missing or if the patient suffers from chronic otitis media (inflammation of the middle ear) or has had a myringotomy (cutting of eardrum to allow fluid to escape from the middle ear). If irrigation cannot be used or fails to remove the cerumen, patients are referred to an ear, nose, and throat (ENT) specialist, who is typically better suited to remove the ear wax with a vacuum device or a curette.

Ear drops to soften ear wax, such as Cerumenex® (triethanolamine polypeptide oleate (otic)), are sometime utilized. Over-the-counter wax removal products include Debrox® (Carbamide Peroxide Otic Solution) or Murine Ear Drops (Carbamide Peroxide). A 3% solution of hydrogen peroxide may also be used. These products are less likely to irritate the skin of the ear.

However and despite these approaches, there still remains a need for simple and long-lasting methods by which excessive ear wax build-up and associated disorders can be treated and/or prevented.

### SUMMARY

Presently descibed are methods useful in treating, that is, alleviating, outer ear conditions generally associated with the secretion of cerumen. The methods comprise the step of locally administering a therapeutically effective amount of neurotoxin such as a botulinum toxin to the outer ear of a patient. The conditions to be treated are generally associated with excess secretion of cerumen or the build-up of cerumen in the ear canal of a patient. Such conditions can include, but are not limited to, conductive hearing loss, pain and cough.

In one aspect, a method for treating excessive cerumen secretion in a patient in need thereof comprises the step of locally administering a therapeutically effective amount of a botulinum toxin to or at least a portion of an ear canal surface of the patient, thereby treating the excessive cerumen secretion. Such a method can be utilized where excessive cerumen secretion results in conductive hearing loss. In a particular example, excessive cerumen secretion elicits pain as a result of the excessive build-up of cerumen in at least one ear canal of a patient, such as a human patient. The botulinum toxin is selected from the group consisting of types A, B, C₁, D, E, F and G. In some examples, the utilized botulinum toxin is botulinum toxin type A and is administered in an amount, for example of between about 1 to about 3000 units or any range or amount therebetween. The botulinum toxin to be utilized in accordance with the teaching of the present disclosure can also be botulinum toxin type B, typically administered at between about 50 units and about 20,000 units or any range or amount therebetween. The methods disclosed herein utilize the local administration of a botulinum toxin, such as, transdermal, subcutaneous, intradermal and intramuscular administration. In particular embodiments, the botulinum neurotoxin can be administered in an amount between about 0.01 U/kg and about 35 U/kg, for example.

Disclosed herein are methods for reducing cerumen secreted in at least a portion of an ear canal of a patient in need thereof, comprising the step of locally administering a therapeutically effective amount of a botulinum toxin to least a portion of an ear canal of the patient, thereby reducing the amount of cerumen secreted. In particular examples, the botulinum toxin type A is administered at between about 1 unit and about 2500 units administered via transdermal, subcutaneous and intradermal administration methods. Some patients may require steps such as irrigating the ear canal of the patient and/or administration of an antibiotic to the ear canal, in addition to the local administration of a therapeutically effective amount of a botulinum neurotoxin. Exemplary antibiotics that can be utilized in accordance with the teachings disclosed herein include, but are not limited to, neomycin, polymyxin B, and hydrocortisone, for example.

Also disclosed herein are methods for treating conductive hearing loss. In particular examples, methods for treating conductive hearing loss in a patient in need thereof comprise the step of locally administering between about 1 unit and about 2000 units of botulinum toxin type A or from about 50 to about 10,000 units of a botulinum toxin type B, to at least a portion of an ear canal of the patient, to thereby treat the conductive hearing loss, which can be a result of excessive build up of cerumen.

Additional embodiments include a method for reducing outer ear pain as a result of excess cerumen build-up in at least a portion of an ear canal of a patient in need thereof. The method in one example can comprising the step of locally administering between about 1 unit to about 500 units, or from between about 5 units to 100 units of botulinum toxin type A to an ear canal of said patient, to thereby reduce the outer ear pain. Botulinum toxin utilized in accordance with the teachings herein disclosed can also be administered to an ear canal to treat a cough, as described herein.

About: as used herein “about” means approximately or nearly and in the context of a numerical value or range set forth means ±15% of the numerical value range recited or claimed.

Enhancing agent: as used herein “enhancing agent” refers to an agent that enhances the permeability of a patient's skin so that botulinum toxin can be absorbed by the skin to achieve a therapeutic effect. In reference to the disclosure herein, enhancing agent can include dimethylsulfoxide (DMSO) or a combination of pluronic lecithin organizer (PLO) and DMSO. An enhancing agent may include, and is not limited to, alcohols, such as short chain alcohols, long chain alcohols, or polyalcohols; amines and amides, such as urea, amino acids or their esters, amides, AZONE® (n-dodecyl-caprolactam), derivatives of AZONE®, pyrrolidones, or derivatives of pyrrolidones; terpenes and derivatives of terpenes; fatty acids and their esters; macrocyclic compounds; tensides; or sulfoxides other than dimethylsulfoxide, such as, decylmethylsulfoxide; liposomes; transfersomes; lecithin vesicles; ethosomes; water; surfactants, such as anionic, cationic, and nonionic surfactants; polyols; and essential oils.

“Alleviate” as applied to pain means a reduction of pain. In some embodiments, the reduction of pain is reduced by more than 25%. In some embodiments, the reduction of pain is by more than 50%. The reduction of pain is measured by the patient reporting the degree of pain, such as on a visual analog scale (VAS) after the neurotoxin treatment, as compared to the degree of pain prior to the treatment. “Alleviate” as applied to cerumen secretion means a reduction of secretion/deposition (for example, an increase of that amount of time taken for excessive cerumen secretion to build-up sufficiently to result in an undesired outer ear condition and/or no observable build-up of cerumen) after neurotoxin treatment as compared to cerumen secretion prior to the neurotoxin treatment. “Alleviate” as applied to conductive hearing loss relates to the increased ability of a patient to hear, after neurotoxin treatment as compared to before neurotoxin treatment. Thus, alleviating includes some reduction, significant reduction, near total reduction, and total reduction of a symptom associated with excessive cerumen secretion, conductive hearing loss, pain, infection and cough, as herein disclosed.

Excessive cerumen secretion: as used herein “excessive cerumen secretion” is an amount of cerumen in a person that results in an associated condition, such as infection, ear pain, conductive hearing loss, or coughing, for example.

Local administration: as used herein “local administration” or “locally administering” means direct administration of a pharmaceutical at or to the vicinity of a site on or within an animal body, at which site a biological effect of the pharmaceutical is desired. Local administration excludes systemic routes of administration, such as intravenous or oral administration, but includes transdermal, subdermal, intradermal and subcutaneous administration. Topical administration is a type of local administration in which a pharmaceutical agent is applied to a person's skin. Topical administration of a neurotoxin, such as botulinum toxin, excludes systemic administration of the neurotoxin. In other words, and unlike conventional therapeutic transdermal methods, topical administration of botulinum toxin does not result in significant amounts, such as the majority of, the neurotoxin passing into the circulatory system of the patient.

Neurotoxin: as used herein “neurotoxin” means a biologically active molecule with a specific affinity for a neuronal cell surface receptor. Neurotoxin includes Clostridial toxins, such as Clostridial botulinum toxins, both as pure toxin (having a molecular weight of about 150 kDa) and as complexed with one or more non-toxin, toxin associated proteins; the complexes having molecular weights of about 900 kD, 700, kD, 500 kD or 300 kD, for example. Botulinum toxins can include toxins that are recombinantly made and modified in accordance with known molecular techniques, that is, a modified neurotoxin means a neurotoxin which has had one or more of its amino acids deleted, modified or replaced (as compared to the native neurotoxin) and includes neurotoxins made by recombinant technology as well as derivatives and fragments of a native or recombinantly produced neurotoxin.

Outer Ear: as used herein “outer ear” means the portion of the ear including the pinna and the ear canal up to the eardrum.

Therapeutically effective: as used herein “therapeutically effective” means an amount of toxin administered that will reduce or ameliorate a condition or symptom (in frequency and/or intensity) resulting from excessive secretion and/or build-up of cerumen in the ear canal of a patient. The therapeutically effective amount of toxin, such as a botulinum neurotoxin, delivered to a patient, is an amount that does not result in undesirable systemic side effects associated with systemic neurotoxin poisoning, as known by those of ordinary skill in the art, but rather elicits a reduction in cerumen secretion, and thus problems associated with its excess, such as conductive hearing loss, pain and cough, for example.

### DETAILED DESCRIPTION

Presently described are methods useful in treating outer ear conditions generally brought on and associated with undesirable (e.g. excessive) secretion of cerumen (commonly known as earwax). The methods comprise the step of locally administering a therapeutically effective amount of neurotoxin such as a botulinum neurotoxin toxin to the outer ear. In one embodiment, the botulinum toxin is administered to the ear canal. The conditions to be treated are generally associated with excess secretion of cerumen and/or the build-up of cerumen in the ear canal of a patient. Such conditions can include, but are not limited to conductive hearing loss, pain and cough.

The following embodiments are non-limiting exemplary situations wherein the methods described herein can be useful. In one embodiment, the methods can be used to treat build-up of cerumen resulting from the use of a hearing aid, wherein the hearing aid blocks the natural removal of cerumen from the outer ear thereby leading to a build-up and/or blockage of the ear canal. In another embodiment, the excessive secretion of cerumen can be a result of ear surgery or trauma to the ear canal. In one embodiment, a build-up of cerumen can result from improper ear canal cleaning with or without excessive cerumen secretion. In particular examples and for particular patients, the local administration of a botulinum toxin can be utilized in conjunction with, or instead of typical ear wax clearing methodologies, as described herein for example, or when such methods (such as mechanical removal of ear wax buildup) is found by a medical practioner to insufficiently address a patients' condition.

The build-up of cerumen in the ear canal can lead to partial or complete blockage of the ear canal. Such a blockage can lead to conductive hearing loss, pain and/or cough. Conductive hearing loss results from cerumen build-up in the ear canal thereby blocking sound waves from reaching the eardrum. In such a case, the cerumen acts in a manner similar to that of an earplug. In other instances, the cerumen can become very hard and brittle, and exert pressure on the sensitive lining of the ear canal. This pressure commonly elicits acute pain in a person's ear. Finally, the ear canal shares some of the same nerves as the throat, so sensations in the ear due to the build-up of cerumen can lead to an itching sensation in the throat, thereby leading to a cough.

Regardless of the cause and/or circumstance resulting in the excess secretion and/or build-up of cerumen, commonly, before the methods described herein can be performed, the existing cerumen should be removed from the patient's ear canal. There are several methods known in the art for removing cerumen from the ear canal; some methods such as, but not limited to, candling are not advised by medical professionals. Additionally, the removal of cerumen, especially excess cerumen, by someone other than a skilled medical professional is not advised as the condition could be worsened, could lead to infection or the patient could puncture the eardrum, all of which can lead to serious complications. Typically, cerumen removal by a medical professional is determined on a case-by-case basis, and depending on the severity of the cerumen build-up, entails hydrating the ear canal, use of a curette or similar device, mineral oils, binocular microscopes, microprobes, suction, and/or combinations thereof.

Methods of administration to the ear canal of a patient can include virtually any method of local administration known to those of ordinary skill in the art. In one embodiment, the botulinum toxin can be delivered topically to the ear canal of a patient. In another embodiment, the botulinum toxin can be delivered subdermally to the ear canal of a patient. In another embodiment, the botulinum toxin can be delivered intradermally to the ear canal of a patient. In another embodiment, the botulinum toxin can be delivered via a slow release implant to the ear canal of a patient (exemplary implants are described in U.S. Pat. Nos. 6,306,423, 6,312,708, exemplary transdermal use of botulinum toxin is discussed in, e.g., U.S. Pat. No. 7,384,918 and U.S. Published Patent Applicaiton No. 20040009180, filed Jul. 11, 2002, all herein incorporated by reference). Other methods of local administration of botulinum toxin are known in the art and are considered within the scope of the present disclosure.

Although one exemplary composition may only contain a single type of neurotoxin, such as botulinum toxin type A, as the active ingredient to suppress cerumen production, other therapeutic compositions may include two or more types of neurotoxins, which may provide enhanced therapeutic effects of the disorders. For example, a composition administered to a patient may include botulinum toxin type A and botulinum toxin type B. Administering a single composition containing two different neurotoxins may permit the effective concentration of each of the neurotoxins to be lower than if a single neurotoxin is administered to the patient while still achieving the desired therapeutic effects.

In one embodiment, the neurotoxin can be administered transdermally. In certain embodiments wherein the neurotoxin is administered transdermally, an enhancing agent can be used to enhance the permeability of the skin thereby allowing the bioactive neurotoxin to act at a desired target structure. An enhancing agent used in combination with the neurotoxin in the pharmaceutical composition can be a DMSO or non-DMSO based enhancing agent. The enhancing agent preferably does not injure the skin, and more preferably, temporarily permeabilizes the skin so that once the neurotoxin has been delivered through the skin, the skin reduces its permeability to other factors. As an example, Catz et al., in U.S. Pat. No. 5,238,933 (herein incorporated by reference), discloses skin permeation enhancer systems which increase the permeability of the skin to transdermally administered, pharmacologically active agents.

In one embodiment, the enhancing agent can be an alcohol. Examples of alcohols include short chain alcohols, such as alcohols having between about 2-5 carbon atoms. Some short chain alcohols include methyl alcohol, ethyl alcohol, n-propyl alcohol, isopropyl alcohol, n-butyl alcohol, isobutyl alcohol, sec-butyl alcohol and t-butyl alcohol, or combinations thereof. The alcohols may be mixed in the composition so that the concentration of alcohol in the composition is between about 5% and 50%, or 10% and 40%, or 20% and 30%. The alcohol may be admixed with glycerin to reduce potential irritation caused by higher concentrations of alcohol. Long chain alcohols are also useful to enhance the transdermal administration of neurotoxins, such as botulinum toxins. Examples of long-chain alcohols include, but are not limited to alcohols having between about 8 to 12 carbon atoms, for example. Some specific examples include the following: 1-hexanol, 1-heptanol, 1-octanol, 1-nonanol, 1-decanol; 1-dodecanol, 2-octanol, 2-decanol, 4-octanol; 5-nonanol; 3-hexen-1-ol; 3-decen-1-ol; and 9-decen-1-ol. Polyalcohols may also be used with the neurotoxin. Examples include propylene glycol, glycerol, polyethylene glycol, and dexpantheol, and combinations thereof.

In another embodiment, an enhancing agent can be a vesicle that is able to store the neurotoxin within the vesicle. The vesicle can diffuse through the skin and thereby deliver the neurotoxin to the target site. The vesicle may be a lipid vesicle. In one specific embodiment, the neurotoxin is incorporated into a transfersome, which are deformable carriers containing lipids and membrane softeners (e.g., Hofer et al., New Ultradeformable Drug Carriers for Potential Transdermal Application of Interleukin-2 and Interferon-.alpha.: Theoretic and Practical Aspects, World J. Surg. 24, 1187-1189 (2000); and U.S. Pat. No. 6,165,500 herein incorporated by reference). Surprisingly, it has been discovered that transfersomes sufficiently transport neurotoxins, including botulinum toxin complexes, across the skin to achieve a therapeutic effect. In other words, the neurotoxin is able to be delivered to a target site and still be bioactive after diffusing through the skin. Capric, lauric, and myristic acid can also be used as skin penetration enhancers.

The botulinum toxins described herein can be incorporated into any topical formulation known in the art. Preferably, the compositions can more easily allow the application of the botulinum toxin into the inner ear of a patient. Suitable compositions include, but are not limited to creams, lotions, hydrogels, jellies, sprays, pastes, adhesives, emulsions, nanoparticles, microparticles, drops, powders, and combinations thereof.

The compositions of the invention may be used in an application device that permits application of the composition to a target site in the ear canal without applying the composition to non-target site areas of the skin. For example, a device may be employed that allows the composition to be applied without first applying the composition to one's fingers, which may lead to undesirable paralysis of the fingers. Suitable devices include spatulas, swabs, syringes without needles, and small adhesive patches. Use of spatulas or swabs, or the like, may require the device to be inserted into a vessel containing the composition then topically spread over the ear canal. Syringes filled with the composition may be used to deposit the composition onto/into the skin of the ear canal.

In one embodiment, the botulinum toxin is dispersed in a composition comprising a cream, lotion or jelly along with an enhancing agent such as an alcohol. The composition can be dispersed on a swab by immersing the swab in the composition. The swab can then be inserted into the ear canal of a patient and applied to the skin therein.

In another embodiment, the botulinum toxin can be associated with a prosthesis that can be placed in the ear canal of a patient and thereby slowly release a botulinum toxin into the skin of the ear canal. In one embodiment, the prosthesis assumes the shape of the outer ear. The botulinum toxin can be impregnated into a polymer matrix associated with the surface of the prosthesis or can be impregnated into the prosthesis itself. The polymer or prosthesis can be tailored to release the botulinum toxin over a predetermined amount of time. Additionally, the prosthesis can further comprise an enhancing agent.

Non-chemical methods of enhancing botulinum toxin release in subdermal structures may include steps of disrupting the stratum corneum to reduce the impermeability of the stratum corneum, and applying a botulinum toxin to the skin location in which the stratum corneum has been disrupted. Disrupting the stratum corneum refers to either completely removing the stratum corneum from a region of a patient's skin, such as in an ear canal, or partially removing portions of the stratum corneum at a location on the patient's skin so that relatively small stratum corneum-free regions of skin are present. The skin may be disrupted using any suitable method without imparting significant pain to the patient. In preferred embodiments of the methods, the stratum corneum is non-chemically disrupted. For example, the stratum corneum may be abrasively scrubbed to disrupt the laminar barrier of the stratum corneum. Or, the stratum corneum may be disrupted by applying an adhesive, such as adhesive tape or wax, to the skin, and subsequently removing the adhesive from the skin. Because such methods of disrupting the stratum corneum may cause some pain, it may be desirable to provide a topical anesthetic to the skin, such as lidocaine cream, to temporarily reduce any pain that may be caused by the disruption. These methods require a skilled medical professional because the skin lining the ear canal is very sensitive, a very compact area, and the procedures may be uncomfortable for a patient.

Additional transdermal methods that non-chemically enhance the skin's permeability include low frequency ultrasound (20 kHz to 1 MHz). Ultrasound is defined as sound at a frequency of between about 20 kHz and 10 MHz, with intensities of between 0 and 3 W/cm². Low frequency ultrasound, as used herein, refers to ultrasound at a frequency that is less than 1 MHz, and preferably in the range of 20 kHz to 40 kHz. The ultrasound is delivered in pulses, for example, 100 msec pulses at a frequency of 1 Hz. The intensity of the ultrasound may vary between 0 and 1 W/cm², and frequently varies between 12.5 mW/cm² and 225 mW/cm². Typical duration of exposure to ultrasound is between about 1 and about 10 minutes. The ultrasound is applied without causing an increase in skin temperature greater than about 1 degree Celsius. Low frequency ultrasound may be used alone or in combination with the composition to improve the permeability of the skin to the neurotoxin. Examples of ultrasound techniques for improving skin permeability may be found in U.S. Pat. Nos. 6,002,961 and 5,814,599, hereby incorporated by reference.

It has been discovered that low frequency ultrasound temporarily disrupts the stratum corneum so that subsequent topical application of botulinum toxin achieves a therapeutic effect. In other words, the disruption caused by the ultrasound persists for several minutes, for example between about 10 and 30 minutes, to provide relatively easy transdermal delivery of botulinum toxin to the patient. After about 30 minutes, the stratum corneum begins to resume its natural structure, and the permeability of the stratum corneum temporally decreases. Thus, one method of the invention, includes the step of applying low frequency ultrasound to one or more regions of the ear canal skin, and subsequently topically applying botulinum toxin to those regions of the skin that were exposed to the low frequency ultrasound, where the botulinum toxin is provided in a composition containing an enhancing agent, which facilitates penetration of the botulinum toxin to the patient.

Additionally, the ultrasound may be delivered prior to application of the botulinum toxin to the skin, or the removal of the cerumen build-up. In fact, the ultrasound may aid in the removal of cerumen as it may loosen more brittle cerumen, hence allowing easier removal.

The neurotoxin may be topically administered by any suitable method as determined by the attending physician. The methods of administration permit the neurotoxin to be administered locally to the ear canal. Methods of administration include coating the skin with the composition so that the composition covers at least a portion of the target site.

The botulinum toxin can further be injected into the patient's ear canal in one or more locations. The injection can be subdermal, subcutaneous or intradermal. The pattern of injections, number of injections, injection sites, amount of toxin per injection site, for example, can be determined on a case-by-case basis by physician, as typically known in the medicinal arts relating to the use therapeutic use of botulinum toxins for treatment of various neuromuscular conditions.

Although the causes and mechanisms of excess cerumen production in the outer ear are not well known, and without wishing to be bound by theory, the Applicant postulates that a botulinum toxin can be used to induce denervation and thereby treat conditions associated with excess cerumen production by inhibiting or preventing excess secretion of cerumen by the cerumen glands in the outer ear. This denervation can result from a reduction in the release of various neuropeptides/neurotransmitters by nerves.

The botulinum toxins used herein inhibit at least a portion of the secretion of cerumen in the outer ear of a patient. The suppressive effects provided by the toxin can persists for several months, such as from about 1 month to about 12 months, or from about 1 month to about 6 months. In one embodiment, the suppression can last for years, for example up to about 2 years.

Exemplary, commercially available, botulinum toxin containing compositions include, but are not limited to, BOTOX® (Botulinum toxin type A neurotoxin complex with human serum albumin and sodium chloride) available from Allergan, Inc., of Irvine, Calif. in 100 unit vials as a lyophilized powder to be reconstituted with 0.9% sodium chloride before use), DYSPORT® (Clostridium botulinum type A toxin haemagglutinin complex with human serum albumin and lactose in the formulation), available from Ipsen Limited, Berkshire, U.K. as a powder to be reconstituted with 0.9% sodium chloride before use) which can be used at about 3 to about 4 times the amounts of BOTOX® as set forth herein in each instance, and MYOBLOC® (an injectable solution comprising botulinum toxin type B, human serum albumin, sodium succinate, and sodium chloride at about pH 5.6, available from Solstice Neurosciences, Inc., South San Francisco, Calif.) which can be used at about 30 to about 50 times the amounts of BOTOX® as set forth herein in each instance, as known in the art. XEOMIN® (a 150 kDa botulinum toxin type A formulation available from Merz Pharmaceuticals, Potsdam, Germany) is another useful neurotoxin which can be used at about 1 to about 2 times the amounts of BOTOX® as set forth herein in each instance.

The amount of toxin administered according to a method within the scope of the present disclosure can vary according to the particular characteristics of the ear condition being treated, including its severity and other various patient variables including size, weight, age, and responsiveness of the particular patent to the botulinum neurotoxin therapy. To guide the practitioner, typically, no less than about 5 units and no more than about 500 units of a botulinum toxin type A (such as BOTOX®) is administered per injection site (i.e. to each ear canal), per patent treatment session. For topical applications, more toxin can be used. For a botulinum toxin type A such as DYSPORT®, preferably no less than about 10 units and no more about 2000 units of the botulinum toxin type A are administered per administration or injection site, per patent treatment session. For a botulinum toxin type B such as MYOBLOC®, preferably no less than about 200 units and no more about 25000 units of the botulinum toxin type B are administered per administer or injection site, per patent treatment session. Less than about 5, 10 or 200 units (of BOTOX®, DYSPORT® and MYOBLOC® respectively) may fail to achieve a desired therapeutic effect, while more than about 500, 2000 or 25000 units (of BOTOX®, DYSPORT® and MYOBLOC® respectively) may result in clinically observable side effects which can vary depending on administration method, site and particular patient. For example and in particular embodiments, an implant that slowly releases a therapeutically effective amount of botulinum toxin can obviously contain an amount of toxin (i.e. of units) that may be higher than an amount that is typically administered, directly (e.g., by subdermal injection). As an illustrative example, while 2000 units of BOTOX® may not be desired to be administered at one time to a target ear canal via a syringe, yet these same 2000 units, when incorporated into a slow-release implant that is placed subdermally in the ear canal, can provide slow, long term dosing/release of botulinum neurotoxin in therapeutically effective amounts.

In additional embodiments, no less than about 10 units and no more about 400 units of BOTOX®; no less than about 30 units and no more than about 1600 units of DYSPORT®, and; no less than about 250 units and no more than about 20000 units of MYOBLOC® are administered per site, per patent treatment session.

In still further embodiments, no less than about 20 units and no more about 300 units of BOTOX®; no less than about 60 units and no more than about 1200 units of DYSPORT®, and; no less than about 1000 units and no more than about 15000 units of MYOBLOC® are administered per site, per patent treatment session. There can be multiple injection sites (i.e. a pattern of injections) for each patient treatment session in order to distribute the neurotoxin over a desired target area, such as around/throughout the ear canal and up to the tympanum.

Although examples of routes of administration and dosages are provided, the appropriate route of administration and dosage are generally determined on a case-by-case basis by the attending physician, as known in the botulinum toxin arts, and titration of the dosage to a therapeutically effective one, for a particular patient/condition, is routinely undertaken. Such determinations are routine to one of ordinary skill in the art (see for example, Harrison's Principles of Internal Medicine (1998), edited by Anthony Fauci et al., 14th edition, published by McGraw Hill). For example, the route and dosage for administration of a Clostridial neurotoxin according to the present disclosed invention can be selected based upon criteria such as the solubility characteristics of the neurotoxin chosen as well as the intensity and scope of the ear condition to be treated.

Additionally, in some embodiments, a physician may have to alter dosage in each case (i.e. patient) in accordance with the assessment of the severity of the condition, as typically done when treating patients with a condition/disorder. Further, in some embodiments, the treatment may have to be repeated at least one additional time, in some cases several times, depending on the severity of the condition and the patient's overall health. If, for example, a patient is not deemed physically suitable for a full administration of botulinum toxin, or if a full administration is not desired for any reason, smaller doses on multiple occasions may prove to be efficacious. Further still, if botulinum toxin is administered at a certain dosage that is not sufficient to attain the desired treatment goal, such as reduction in an amount of ear wax produced over a particular time period, the dose may be increased for a second and subsequent administration session(s) by the attending physical as he/she sees fit.

It is understood that cerumen can be a natural defense the body has to protect the eardrum. As such, a therapeutically effective amount of a botulinum toxin used to reduce the secretion of cerumen can be an amount which is less than that needed to completely eliminate cerumen secretion. In particular embodiments, eliminating secretion of cerumen may in fact be efficacious, such as in patients wearing hearing aids wherein the hearing aids act as a dam to natural cerumen removal. A skilled physician can easily determine the dose of botulinum toxin required to attain a desired outcome, and as such, treats patients on a case-by-case basis using the general guidelines taught herein.

A further benefit of the methods described herein is a reduced chance of middle and inner ear infection due to patient self remedy steps. Patients, such as those that have had ear trauma resulting in tears or holes in the eardrum run an increased risk of inner ear infection. Further, many home remedies for cerumen build-up require the hydration of the ear canal which can be extremely problematic for people with tears or holes in their eardrums, as those holes or tears can make the inner ear susceptible to the elements. The use of a method such as those described herein for people with tears or holes in their eardrums can reduce their reliance on home remedies to treat excess cerumen production and/or build-up and hence reduce their chances of developing an inner ear infection as a result of utilizing a home remedy.

In one embodiment, a barrier device can be inserted into the ear canal by a trained physician up to the point of the eardrum, thereby sealing the eardrum off from the outside environment. This method can be beneficial for those with a hole or tear in the eardrum. The barrier device can be a plug made of any acceptable substance such as wax, polymer, silicone-rubber or the like. After the eardrum is thus isolated, the physician can use any method of transdermal administration described herein. For example, and not intended as a limitation, drops containing botulinum toxin can be dropped into an ear with the patients head sideways with the ear canals substantially vertical. The drops can be allowed to sit for an appropriate amount of time (such as up to about one hour, preferably up to about 30 minutes, more preferably up to about 15 minutes) and then removed either by suction, absorption onto a cotton swab or fabric or gravity (shaking the drops out). After the drops have thus been administered and the residual removed, the physician can then carefully remove the barrier device.

Further, in some embodiments, it is advantageous for a physician to utilize an ear, nose and throat (ENT) microscope or similar device to aid in properly removing cerumen and properly applying the botulinum toxin to the effected area of the ear canal. In some embodiments, particular spots or sections of the ear canal can have botulinum toxin topically applied or injected. Injection into certain sections of the ear canal may require the use of an ENT microscope or equivalent device, to administer the botulinum neurotoxin in the desired amount at the desired locations.

### EXAMPLE 1

A 52 year old woman weighing 42 kg comes into her ear, nose and throat (ENT) specialist's office complaining of ear pain in the left ear. She is a fit and active woman, yet is a habitual smoker. Upon examination of the patient's left ear, the ENT specialist observes a build up of cerumen in the outer ear of the patient. The ENT specialist lubricates the outer ear with warm water to loosen the cerumen, and thereafter carefully removes the cerumen with a curette. The ENT specialist then coats a cotton swab with 5 units of botulinum toxin type A suspended in a cream. The cream is spread thinly on the surface of the ear canal of the patient. The patient is instructed not to touch the ear for at least five hours.

The patient returns for a follow-up visit with the ENT specialist four months later still complaining of pain in the left ear. Upon examination, the specialist notices a decrease in cerumen build-up in the outer ear, however, the amount of cerumen remains unacceptable. The ENT specialist again lubricates and carefully removes the cerumen from the patient's outer ear. The ENT specialist now applies about 20 units of botulinum toxin type A (such as BOTOX® or about 80 units of DYSPORT®) suspended in cream to the outer ear surface of the patient. The patient is again instructed not to touch the ear for at least five hours.

Four months later, the patient returns for a follow-up visit and upon examination of the left ear, the ENT specialist notes that there is no unwanted/excessive build up of cerumen in the outer ear and accordingly the patient notes that she does not experience any pain in the left ear.

### EXAMPLE 2

A 72-year-old male weighting 150 kg, suffering from diabetes, liver disease, meningitis, atherosclerosis and tuberculosis, and who wears hearing aids in both ears visits his geriatrician complaining that he has lost hearing in both ears despite using the hearing aids. The doctor carefully removes the hearing aids from both ears and checks the batteries. Noting that the batteries appeared to work sufficiently, the doctor examines the ears of the patient. Upon examination, the doctor notes significant cerumen build-up in the right ear and mild build-up in the left ear. The doctor lubricates both ears and carefully removes the cerumen with a curette. The doctor then coats a cotton swab with about 20 units of botulinum toxin type A suspended in a cream. The cream is spread thinly on the surface of the left ear canal of the patient. The patient is instructed not to touch the ear for at least five hours. The doctor then coats a cotton swab with about 35 units of botulinum toxin type A suspended in a cream. The cream is spread thinly on the surface of the right ear canal of the patient. The patient is instructed not to touch the ear for at least five hours.

Four months later, the patient returns for a follow-up visit and upon examination of the left ear, the doctor notes that there is no build up of cerumen in either ear. The patient notes that he has not experienced any loss of hearing since the last treatment and is now able to hear sounds and voices utilizing the same hearing aids. This example shows that differing ears, even on the same patient, may require different dosing of botulinum toxin to achieve a therapeutic effect.

### EXAMPLE 3

A 23 year old woman weighing 50 kg comes into her primary care physician's office complaining of ear pain in the left ear. She is a fit and active woman. Upon examination of the patient's left ear, the physician observes a build up of cerumen in the outer ear of the patient. The physician lubricates the outer ear with warm water to loosen the cerumen, and thereafter, carefully removes the cerumen from the ear canal with a curette. The physician then injects 30 units of botulinum toxin type A (such as BOTOX®) into various intradermal locations in the patient's left ear canal. The patient is instructed not to touch the ear for at least five hours.

Four months later, the patient returns for a follow-up visit and upon examination of the left ear, the physician notes that there is no build up of cerumen in the left ear. The patient notes that she has not experienced any pain in her left ear since the treatment. After a year and at her next visit to the primary care physician, the patient reports that she is free of pain in her left ear.

### EXAMPLE 4

A 80 year old woman weighing 40 kg comes into her ENT specialist's office complaining of ear pain in the right ear. She has been previously diagnosed with a tear in her right eardrum and has experienced countless inner ear infections due to home remedy cerumen removing procedures. Upon examination of the patient's right ear, the physician observes a build up of cerumen in the outer ear of the patient. The physician images the ear canal with an ENT microscope and proceeds to carefully loosen the cerumen, and thereafter, carefully removes the cerumen with a curette. The physician then inserts a wax barrier device into the patient's ear. The doctor then coats a cotton swab with 500 units of botulinum toxin type B (e.g. MYOBLOC® botulinum toxin type B) suspended in a cream. The cream is spread thinly on the surface of the right ear canal of the patient. The cream is allowed to sit for 15 minutes after which the wax barrier device is removed from the patient's ear canal. The patient is instructed not to touch the ear for at least five hours.

Three months later, the patient returns for a follow-up visit and upon examination of the right ear, the physician notes that there is no build up of cerumen in the right ear. The patient notes that she has not experienced any pain in her right ear since the treatment and she has not felt the need to use an at home remedy for cerumen removal.

### EXAMPLE 5

A 23 year old man weighing 80 kg who is a world decathlon champion, comes into his ENT specialist's office complaining of ear pain in the right ear. Upon examination of the patient's right ear, the ENT specialist observes a build up of cerumen in the outer ear of the patient. The ENT specialist lubricates the outer ear with warm water to loosen the cerumen, and thereafter, carefully removes the cerumen with a curette. Upon further examination using an ENT microscope, the ENT specialist determines that the excessive secretion is a result of secretion of the lower quadrant of the ear canal. The ENT specialist decides that a topical administration of botulinum toxin type A/type B mixture to the lower quadrant of the ear canal would be the most appropriate treatment. The ENT specialist proceeds to use a small abrasive material (e.g. a small nylon bristle brush) with the aid of the ENT microscope to roughen up the skin in the effected area of the ear canal. A mixture of botulinum toxin type A/type B suspended in a jelly is applied to the effected region and allowed to sit for about five minutes. The patient is instructed not to touch the ear for at least five hours.

Six months later, the patient returns for a follow-up visit and upon examination of the right ear, the ENT specialist notes that there is no build up of cerumen in the right ear. The patient notes that he has not experienced any pain in his right ear since the treatment.

### EXAMPLE 6

A 78-year-old male weighting 100 kg, who wears a hearing aid in the right ear visits his geriatrician complaining that he has recently noticed a loss of hearing in his right ear despite using the hearing aid, which up until this point had bee satisfactory. After checking the hearing aid for mechanical and electrical failures and noting that the hearing aid works sufficiently, the doctor examines the ear canal of the patient. Upon examination, the doctor notes significant cerumen build-up in the right ear canal. The doctor lubricates the right ear canal and carefully removes the cerumen with a curette and radially locally administers (subdermally) about 20 units of botulinum toxin type A, around the ear canal.

Three months later, the patient returns for a follow-up visit and upon examination of the right ear, the doctor notes that there is no build up of cerumen in the ear. The patient notes that he has not experienced any loss of hearing since the last treatment and is now able to hear sounds and voices clearly enough, utilizing the same hearing aid.

### EXAMPLE 7

A 59 year old male presents to his primary physician with a nagging, irritation cough that has been bothering this patient for two days. After investigating the patient's throat, the physician move on to view the patient's ear canals and notes that both canals are over 50% blocked with cerumen. After delicately removing the built-up cerumen utilizing a curette and a warm water/peroxide solution, and irrigating the patient's ear canals, the physician proceeds to apply lidocaine topical anesthetic in advance of injecting, subdermally and in a radial fashion, about 2 units of BOTOX® (botulinum toxin type A complex) at approximately the 12, 3, 6 and 9 o'clock positions of the ear canal for a total of 8 units of BOTOX® (in each canal), after which antibiotic ear drops are administered. During a follow-up visit 5 months later, during which the physician no longer finds cerumen blockage of the ear canal, the patient reports that he is no longer tormented by the previously annoying cough, and that he now can also hear more clearly than before.

### EXAMPLE 8

A 22 year old college student presents to his physician, complaining of ear pain and a noticeable decrease in his ability to hear and understand normal-volume conversation and audio from the television, for example. Upon examination and interview, it is learned that the student had a violent spill on the local ski slopes, banging the left side of his head upon the hard ice (no new snow had fallen for 4 weeks prior to his accident) a month ago. The physician notes the patient's first score on a hearing test utilizing an audiometer, and then proceeds to clear out, what is noted to be excessive cerumen build-up in the left ear canal and administers 10 units of a botulinum toxin type A (5 units of BOTOX® to the top of the ear canal and 5 units to the opposite side of the canal) via a 30 gauge syringe. After 3 months and during a follow up visit, the patient notes that the previously reported ear pain has subsided and that he can hear normal conversation very well once more. Upon testing his hearing, the physician notes that his hearing test score has improved as compared to the first score. A follow up six months later results in similar hearing maintenance and no pain to note.

In one aspect, the present disclosure includes, as one of ordinary skill in the art understands, the use of a botulinum toxin, such as botulinum toxin types A, B, C₁, D, E, F and G, in the manufacture of a medicament for treating excessive cerumen secretion, as well as resultant associated disorders, such as conductive hearing loss, pain, cough, or infection for example.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. For example, while all embodiments described herein are directed to human patients, the teachings of the present disclosure can likewise be utilized by veterinarians to treat excessive cerumen buildup in domesticated mammals, such as a canine or feline patient. Accordingly, the present invention is not limited to that precisely as shown and described.

I claim:

1. A method for treating excessive cerumen secretion in a patient in need thereof, comprising the step of locally administering a therapeutically effective amount of a botulinum toxin to or at least a portion of an ear canal surface of the patient, thereby treating the excessive cerumen secretion.

2. The method according to claim 1 wherein said excessive cerumen secretion results in conductive hearing loss.

4. The method according to claim 1 wherein said excessive cerumen secretion elicits pain as a result of excessive build-up of cerumen in the ear canal of said patient.

5. The method according to claim 1 wherein said botulinum toxin is selected from the group consisting of types A, B, C₁, D, E, F and G.

6. The method according to claim 5 wherein said botulinum toxin is botulinum toxin type A and is administered in an amount between about 1 to about 2500 units.

7. The method according to claim 5 wherein said botulinum toxin type B is administered at between about 50 units and about 20,000 units.

8. The method according to claim 1 wherein said local administration is transdermal administration.

9. The method according to claim 1 wherein said local administration is subcutaneous administration.

10. The method according to claim 1 wherein said local administration is intradermal administration.

11. A method for reducing cerumen secreted in at least a portion of an ear canal in a patient in need thereof, comprising the step of locally administering a therapeutically effective amount of a botulinum toxin to least a portion of an ear canal of the patient, thereby reducing the amount of cerumen secreted.

12. The method according to claim 11 wherein said botulinum toxin is selected from the group consisting of types A, B, C₁, D, E, F and G.

13. The method according to claim 12 wherein said botulinum toxin is botulinum toxin type A or type B.

14. The method according to claim 13 wherein said botulinum toxin type A is administered at between about 1 unit and about 2500 units.

15. The method according to claim 11 wherein said local administration is selected from the group consisting of transdermal, subcutaneous and intradermal administration.

16. The method of claim 11, further comprising the step of irrigating the ear canal of the patient.

17. The method of claim 11, further comprising the step of administering an antibiotic to the ear canal.

18. The method of claim 11, further comprising the step of administering an anesthetic to the ear canal.

19. A method for treating conductive hearing loss in a patient in need thereof, comprising the step of locally administering between about 1 unit and about 2000 units of botulinum toxin type A or about 50 to about 5000 units of botulinum toxin type B to at least a portion of an ear canal of said patient, thereby reducing cerumen secretion and resultant build-up and treating conductive hearing loss.

20. A method for reducing outer ear pain as a result of excess cerumen build-up in at least a portion of an ear canal of a patient in need thereof, comprising the step of locally administering between about 1 unit and about 2000 units of botulinum toxin type A to the ear canal of said patient, thereby reducing the outer ear pain.
